(19) United States
(12) Patent Application Publication
(10) Pub. No.: US 2016/0243011 A1
(43) Pub. Date: Aug. 25, 2016

(54) Formulations Comprising Idebenone, N-Acetyl-S-Farnesyl-L-Cysteine and Ergothioneine and Uses Thereof

(71) Applicant: ELIZABETH ARDEN, INC., New York, NY (US)

(72) Inventors: Jose Fernandez, Monmouth Junction, NJ (US); Eduardo Perez, Monmouth Junction, NJ (US); Maxwell Stock, Monmouth Junction, NJ (US); Lavinia Codruta Popescu, Jackson Heights, NY (US); Aurelie Nathalie Felix-Gonnot, New York, NY (US); Arthur Joseph Pellegrino, Newton, PA (US)

(21) Appl. No.: 14/426,777

(22) PCT Filed: Sep. 11, 2013
(86) PCT No.: PCT/US13/59257

§ 371 Date: Mar. 9, 2015

Related U.S. Application Data
(60) Provisional application No. 61/815,771, filed on Apr. 25, 2013.
(60) Provisional application No. 61/701,098, filed on Sep. 14, 2012.

(51) Int. Cl. A61K 8/37; A61K 8/49; A61Q 17/04; A61Q 19/08; A61K 31/198; A61K 31/4172; A61Q 19/00; A61K 8/44; A61K 31/215

(52) U.S. Cl. CPC A61K 8/37; A61K 8/447; A61K 8/4946; A61Q 17/04; A61K 31/215; A61K 31/198; A61K 31/4172; A61Q 19/004; A61Q 19/08; A61K 2800/70; A61K 2800/596

(57) ABSTRACT

Compositions that mitigate damage caused by environmental stressors are provided. The stressors include, but are not limited to, damage caused by ultra-violet radiation. The compositions comprise an effective amount of idebenone or a derivative thereof, N-acetyl-S-farnesyl-L-cysteine or a pharmaceutically acceptable salt or ester thereof and ergothioneine or a pharmaceutically acceptable salt or ester thereof. In some embodiments these compositions are applied topically to a person's skin.

### CROSS-REFERENCE TO RELATED APPLICATIONS

This application is a national stage application of PCT/US2013/059257, filed Sep. 11, 2013, which claims the benefit of the filing date of both U.S. Provisional Application Ser. No. 61/701,098, filed Sep. 14, 2012 and U.S. Provisional Application Ser. No. 61/815,771, filed Apr. 25, 2013; the entire disclosures of the afore-referenced applications are incorporated by reference as set forth fully herein.

### FIELD OF THE INVENTION

The present invention relates to the field of protection against damage from environmental stressors.

### BACKGROUND OF THE INVENTION

The skin is under constant attack from a variety of environmental stressors such as ultraviolet (UV) radiation from the sun. These stressors can cause damage to the skin that accumulates with age and that can result in chronic inflammation, DNA damage and reactive oxidative stress (ROS).

In order to protect against environmental stressors and to repair damage that these stressors cause, researchers have developed topical cosmetic and dermatologic preparations that contain certain active ingredients. For example, researchers have discovered that idebenone is a powerful antioxidant that protects against skin aging. Because of the benefits of this compound, it is included in the commercially available facial serum Prevage®.

Although many consumers have recognized the benefits of using Prevage®, researchers are always looking for ways to provide new and improved formulations for preventing or remediating harms that environmental stressors cause. The present invention is directed toward this goal.

### SUMMARY OF THE INVENTION

The present invention provides formulations for protecting against skin damage as well as methods for making and using these formulations. Through the various embodiments of the present invention, a person may gain protection against and/or remediate damage that is caused by environmental stressors.

According to a first embodiment, the present invention is directed to a formulation comprising: (1) idebenone or a derivative thereof; (2) N-acetyl-S-farnesyl-L-cysteine or a pharmaceutically acceptable salt or ester thereof; and (3) ergothioneine or a pharmaceutically acceptable salt or ester thereof.

According to a second embodiment, the present invention is directed to a method of preventing a skin change or mitigating damage to the skin comprising administering to an organism (e.g., a human) a formulation comprising: idebenone or a derivative thereof, N-acetyl-S-farnesyl-L-cysteine or a pharmaceutically acceptable salt or ester thereof and ergothioneine or a pharmaceutically acceptable salt or ester thereof.

According to a third embodiment, the present invention provides for use of a composition or formulation in the manufacture of a medicament useful for treating a skin condition or cosmetic need. The medicament may be used in a method for treating, lessening the severity of and/or delaying onset of inflammation in a subject, including a human, in need thereof, comprising the step of administering an effective amount of a formulation comprising idebenone or a derivative thereof, N-acetyl-S-farnesyl-L-cysteine or a pharmaceutically acceptable salt or ester thereof and ergothioneine or a pharmaceutically acceptable salt or ester thereof, a carrier and optionally an additional active ingredient. In a further aspect, the present invention provides a method for treating, lessening the severity of and/or delaying onset of inflammation in a subject, including a human, in need thereof, comprising the step of administering a provided formulation.

Various embodiments of the present invention provide one or more of the following advantages: (1) a synergistic reduction of IL-6 and TNF-α; (2) anti-aging and anti-inflammatory benefits by modulating MMP-1 and IL-6 levels; and (3) protection against UVB induced sun burn cell formation and IL-6 production.

### DETAILED DESCRIPTION OF THE INVENTION

Reference will now be made in detail to various embodiments of the present invention, examples of which are illustrated in the accompanying figures. In the following description, numerous specific details are set forth in order to provide a thorough understanding of the present invention. However, unless otherwise indicated or implicit from context, the details are intended to be examples and should not be deemed to limit the scope of the invention in any way.

According to one embodiment, the present invention is directed to a formulation that might be either cosmetic or dermatologic and that comprises: (1) idebenone or a derivative thereof; (2) N-acetyl-S-farnesyl-L-cysteine or a pharmaceutically acceptable salt or ester thereof; and (3) ergothioneine or a pharmaceutically acceptable salt or ester thereof. The term “formulation” refers to a solution, suspension, cream, ointment, powder or other combination, e.g., a mixture that contains each of the recited ingredients. Further, a formulation may be a solid, a liquid, a gel or a combination thereof.

As a person of ordinary skill in the art will recognize, when one refers to a molecule or a pharmaceutically acceptable salt or ester thereof, the molecule may be present predominantly or exclusively in a pH neutral form, predominantly or exclusively in an acid form (e.g., having a carboxylic acid group), predominantly or exclusively in a basic form, predominantly or exclusively in a salt form or in some combination of the different forms. The amounts of different forms may be determined by standard equilibrium equations, the environmental conditions of a formulation and the other components of the formulation.

The term “pharmaceutically acceptable salt” refers to those salts that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. Such salts can be prepared in situ during the final isolation and purification of the compounds of the invention, or separately (e.g., by reacting the free base functionality with a suitable organic or inorganic acid).

Alternatively or additionally, salts may form during formulation of a compound. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable ester” refers to an ester that hydrolyzes in vivo and includes esters that break down readily in the human body to leave the parent compound or a salt thereof. Suitable ester groups include, for example, those derived from pharmaceutically acceptable aliphatic carboxylic acids, particularly alkanoic, alkenoic, cycloalkanoic, and alkanedioic acids, in which each alkyl or alkenyl moiety advantageously has not more than six carbon atoms. Examples of particular esters include formates, acetates, propionates, butyrates, acrylates, and ethylsuccinates. In certain embodiments, the esters are cleaved by enzymes such as esterases.

Preferably, each component is present in an effective amount. An “effective amount” refers to the amount of the formulation that is required to confer the desired effect on the person to whom it is administered as recognized by those skilled in the art. This amount may depend on the size of the region to which it is administered and the degree to which the person to whom it is administered will be or has been exposed to one or more environmental stressors. For example, in some embodiments, an effective amount may be an amount necessary to provide one or more of: protection against UV-induced inflammation and photo-damage, reduction of IL-6, reduction of TNF-α, anti-aging and anti-inflammatory properties by modulating MMP-1 and IL-6 levels and protection against UVB induced sunburn cell formulation. “MMP-1” refers to matrix metalloproteinase. “IL-6” refers to interleukin 6. “TNF-α” refers to tumor necrosis factor alpha.

A skilled artisan can determine a pharmaceutically effective amount of the inventive compositions by determining the unit dose. As used herein, a “unit dose” refers to the amount of inventive composition required to produce a response of 50% of maximal effect (i.e. ED50). The unit dose can be assessed by extrapolating from dose-response curves derived from in vitro or animal model test systems.

Idebenone is the main cosmetic functional ingredient in Prevage® facial serum, and it has been shown to be a powerful anti-oxidant that can protect against skin aging. It also has anti-inflammatory properties. The term “idebenone” refers to 6-(10-hydroxydecyl)-2,3-dimethoxy-5-methyl-1,4 benzochinol, which is also known as 2-(10-hydroxydecyl)-5,6-dimethoxy-3-methyl-cyclohexa-2,5-diene-1,4-dione. Its chemical structure is depicted below:

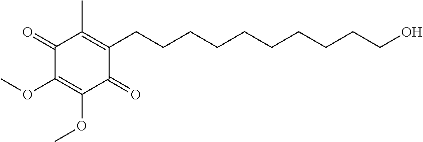 [SMILES: COC1=C(OC)C(=O)C(CCCCCCCCCCO)=C(C)C1=O]

The use of idebenone as part of a topically applied composition is disclosed in U.S. Pat. No. 6,756,045, issued Jun. 29, 2004, the entire disclosure of which is incorporated by reference.

Derivatives of idebenone include, but are not limited to, carboxylic acid substitute derivatives that are defined by the formula below, as well as their salts:

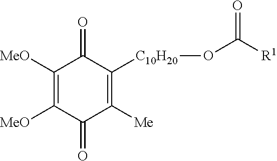

wherein R¹ is a C₂₋₂₂, C₂₋₁₀, C₂₋₅, C₁₄₋₂₀ or C₁₅₋₁₈ straight or branched sugar acid, and wherein two or more hydroxy groups on the sugar acid are each independently substituted with a C₁₋₂₂ carboxylic acid. Preferably, 2, 3, 4, or 5 hydroxy groups of the sugar acid are each independently substituted with a C₁₋₂₂ carboxylic acid. Preferred idebenone compounds of the present invention may also include fewer hydroxy groups substituted with longer chain carboxylic acids or more hydroxy groups substituted with shorter chain carboxylic acids.

Suitable carboxylic acids for use in the present invention include monocarboxylic acids and polycarboxylic acids. The carboxylic acids may be straight chained, saturated carboxylic acids (e.g., formic acid, acetic acid, propionic acid, butyric acid, valeric acid, caproic acid, enanthic acid, caprylic acid, pelargonic acid, capric acid, lauric acid, palmitic acid, and stearic acid) or short-chain unsaturated monocarboxylic acids (e.g., acrylic acid).

Preferably, carboxylic acids of the present invention are fatty acids (e.g., conjugate fatty acids, medium to long-chain saturated and unsaturated monocarboxylic acids, such as docosahexaenoic acid, and eicosapentaenoic acid). Carboxylic acids for use in the present invention also include amino acids, keto acids (e.g., pyruvic acid, acetoacetic acid), aromatic carboxylic acids (e.g., benzoic acid, salicylic acid), dicarboxylic acids (e.g., aldaric acid, oxalic acid, malonic acid, malic acid, succinic acid, glutaric acid, adipic acid, maleic acid, fumaric acid, phthalic acid, etc.), tricarboxylic acids (e.g., citric acid, isocitric acid, aconitic acid, propane-1,2,3-tricarboxylic acid (e.g., tricarballylic acid, carballylic acid)), alpha hydroxycarboxylic acids (e.g., glycolic acid, lactic acid, hydroxyacrylic acid, oxybutyric acid, glyceric acid, malic acid, tartaric acid and citric acid), and hyaluronic acid.

A “sugar acid” is defined as a straight or branched, saturated or unsaturated, substituted or unsubstituted C₂₋₂₂ (preferably C₂₋₁₀, more preferably C₂₋₅) alkyl group substituted with two or more carboxyl groups wherein the hydroxy functional groups of two or more carboxyl groups are each independently substituted with a C₁₋₂₂ carboxylic acid (preferably C₁₄₋₂₀, more preferably C₁₅₋₁₈). The term “branched” refers to one or more lower alkyl groups such as methyl, ethyl, or propyl attached to a linear alkyl chain. Preferably, 2, 3, 4, or 5 hydroxy groups on the sugar acid are each independently substituted with a C₁₋₂₂ carboxylic acid.

One non-limiting example of a derivative of idebenone is idebenone dipalmitoyl glycerate. Methods for making idebenone dipalmitoyl glycerate are disclosed in U.S. Pat. No. 8,173,703, issued May 8, 2012, the entire disclosure of which is incorporated by reference. The chemical formula for this structure is provided below:

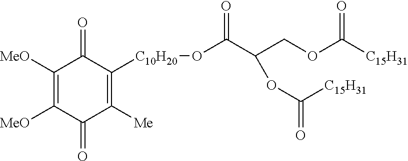

Other derivatives of idebenone may be formed by replacing one or more of its atoms with another atom or group of atoms. For example, one or both of the methoxy groups may be replaced with other alkyl-oxy groups that are unsubstituted or substituted. Additionally, unless otherwise specified or apparent from context, derivatives of idebenone include pharmaceutically acceptable salts and esters of idebenone.

The term ergothioneine refers to 3-(2-sulfanylidene-1,3-dihydroimidazol-4-yl)-2-(trimethylazaniumyl)propanoate. Its chemical structure is depicted below:

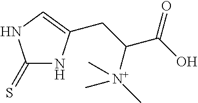

Ergothioneine has free radical scavenging capacity. Thus, it may advantageously be used to remediate damage caused by environmental stressors such as UV radiation and other carcinogens.

N-acetyl-S-farnesyl-L-cysteine, which is commercially available as Arazine™, exhibits anti-inflammatory properties, inhibiting the release of pro-inflammatory mediators and the migration and activation of inflammatory cells. By itself, it can inhibit local inflammation induced by UVA or UVB when applied topically. Its chemical structure is depicted below:

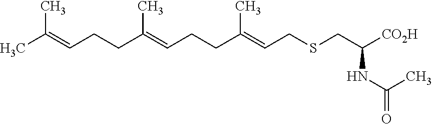 [SMILES: CC(=O)N[C@@H](CSC/C=C(\C)CC/C=C(\C)CCC=C(C)C)C(=O)O]

N-acetyl-S-farnesyl-L-cysteine is a cysteine derivative. As described in U.S. Pat. No. 5,043,268, issued Aug. 27, 1991, the disclosure of which is incorporated by reference, a class of cysteine derivatives of which N-acetyl-S-farnesyl-L-cysteine is a species has the ability to function as a substrate for a specific type of methyltransferase enzymes, the prenyl cysteine methyltransferase enzymes. These enzymes catalyze the transfer of methyl groups from S-adenosylmethionine to the C-terminal carboxylic acid groups of proteins and peptides, including GTP-binding proteins that have a prenylated cysteine residue at their C-termini. N-acetyl-S-farnesyl-L-cysteine inhibits the aforementioned enzymes by functioning as a preferred substrate over the natural substrate. The activity of N-acetyl-S-farnesyl-L-cysteine is further described in US 2010/0247461 A1, published Sep. 30, 2010, the entire disclosure of which is incorporated by reference.

In some embodiments, the N-acetyl-S-farnesyl-L-cysteine is associated with a binding partner in a complex, whereas in other embodiments it is not associated with a binding partner. As used herein, the term “binding partner” refers to an agent that is non-covalently associated with an N-acetyl-S-farnesyl-L-cysteine compound in a complex. In some embodiments, the association between a binding partner and an N-acetyl-S-farnesyl-L-cysteine compound is stable in aqueous solution. In some embodiments, the association between a binding partner and an N-acetyl-S-farnesyl-L-cysteine compound is not stable in aqueous solution. In some embodiments, association between a binding partner and an N-acetyl-S-farnesyl-L-cysteine compound takes the form of a coordination complex. In some embodiments, the binding partner is a metal, a technetium isotope, a small molecule containing a basic nitrogen, a topical analgesic, an opiate, a morphinomimetic, an anti-cancer agent and/or an intraocular pressure reducing agent. Examples of binding partners and complexes are described in paragraphs [0178]-[0222] of U.S. 2010/0247461, published Sep. 30, 2010, which are incorporated by reference.

In some embodiments, the amount of idebenone, or a derivative thereof, is 0.0001-5.0 wt. %, 0.001-5.0 wt. %, 0.01-5.0 wt. %, 0.1-5.0 wt. %, 1.0-5.0 wt. % or 2.0-4.0 wt. % based on the total weight of the formulation of which it is a part.

In some embodiments, the amount of N-acetyl-S-farnesyl-L-cysteine or a pharmaceutically acceptable salt or ester thereof is 0.0001-10.0 wt. %, 0.001-10.0 wt. %, 0.01-10.0 wt. %, 0.1-10.0 wt. %, 1.0-10.0 wt. %, 2.0-8.0 wt. % or 4.0-6.0 wt. % based on the total weight of the formulation of which it is a part.

In some embodiments, the amount of ergothioneine or a pharmaceutically acceptable salt or ester thereof is 0.0001-10.0 wt. %, 0.001-10.0 wt. %, 0.01-10.0 wt. %, 0.1-10.0 wt. %, 1.0-10.0 wt. %, 2.0-8.0 wt. % or 4.0-6.0 wt. % based on the total weight of the formulation of which it is a part.

By way of a non-limiting example, in some embodiments, the weight ratio of idebenone or its derivative to N-acetyl-S-farnesyl-L-cysteine or a pharmaceutically acceptable salt or ester thereof and to ergothioneine or a pharmaceutically acceptable salt or ester thereof is approximately 1:0.5-3:0.5-3, or 1:1.5-3:1.5-3, for example approximately 1:2:2. In other embodiments, the components are present in approximately the same molar ratios or molar ratios of idebenone or its derivative to N-acetyl-S-farnesyl-L-cysteine or a pharmaceutically acceptable salt or ester thereof and to ergothioneine or a pharmaceutically acceptable salt or ester thereof of approximately 1:0.5-3:0.5-3, or 1:1.5-3:1.5-3.

By combining idebenone or a derivative thereof, N-acetyl-S-farnesyl-L-cysteine or a pharmaceutically acceptable salt or ester thereof, and ergothioneine or a pharmaceutically acceptable salt or ester thereof (collectively, the “active ingredients”), one can inhibit UV induction of pro-inflammatory cytokines to a greater degree than one would have expected based on the known activities of each of these compounds.

When idebenone or a derivative thereof, N-acetyl-S-farnesyl-L-cysteine or a pharmaceutically acceptable salt or ester thereof, and ergothioneine or a pharmaceutically acceptable salt or ester thereof, are combined prior to administration to a person, there is a synergistic effect with respect to the response to the damaging effect of UVB. This effect may reduce the expression of IL-6 and TNF-α induced by UVB as compared to a control group of non-irradiated cells. Surprisingly, this effect was not observed when the three compounds were added one by one sequentially to the cells, indicating that these elements, when formulated and applied together demonstrate significantly greater anti-inflammatory properties than when applied separately. The pre-mixing of a blend of these active ingredients may, for example, be applied in a serum that contains one or more if not all of the ingredients in Table 1. The second column of Table 1 recites example weight percentages of the components. The third column provides ranges of these ingredients. A person of ordinary skill in the art will readily appreciate that the broader ranges of the third column include sub-ranges in which the examples of the second column are one of the endpoints, i.e. the lower or upper endpoints of a range the other end of which is defined by one of the endpoints of the third column.

The formulation may also comprise a carrier, such as one that is pharmaceutically acceptable. The carrier is an agent or vehicle for delivering the formulation, and it may be referred to as an excipient. Preferably it is of sufficiently high purity and sufficiently low toxicity to render it suitable for administration. The (pharmaceutical) carrier can be, without limitation, a binding agent (e.g., pregelatinised maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulphate, etc.). Other suitable (pharmaceutical) carriers for the compositions of the present invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Optionally, the formulation comprises a moisturizing agent. As used herein, “moisturizing agent” refers to a substance that adds or restores moisture to the skin or a mucous membrane. Representative examples of moisturizing agents (often referred to as humectants) that are suitable in the present invention include, but are not limited to, guanidine, glycolic acid and glycolate salts, aloe vera in any of its variety of forms, allantoin, urazole, polyhydroxy alcohols such as sorbitol, glycerol, hexanetriol, polypropylene glycol, butylene glycol, hexylene glycol and the like, polyethylene glycols, sugars and starches and their derivatives, hyaluronic acid, lactamide monoethnolamine, acetamide mono ethanol amine, and any combination thereof.

Also optionally, the formulation may comprise a fragrance. As used herein, “fragrance,” refers to a substance having a pleasant aroma. Suitable fragrances include, without limitation, eucalyptus oil, camphor synthetic, peppermint oil, clove oil, lavender, chamomile, and the like.

As noted above, synergistic results were observed when the idebenone or its derivative, N-acetyl-S-farnesyl-L-cysteine or a pharmaceutically acceptable salt or ester thereof and ergothioneine and a pharmaceutically acceptable salt or ester thereof were pre-mixed, and thus part of a “pre-mix blend,” i.e., at least all of the active ingredients are combined prior to application to a cell or to an organism. When creating a pre-mix blend, one may combine all of idebenone or its derivative, N-acetyl-S-farnesyl-L-cysteine or a pharmaceutically acceptable salt or ester thereof and ergothioneine and a pharmaceutically acceptable salt or ester thereof at the same time, or one may first combine two of the ingredients and then combine the third of the ingredients with the first two. Additionally, all of the serum ingredients or other additional components may be pre-combined and then combined with a mixture of idebenone or a derivative thereof, N-acetyl-S-farnesyl-L-cysteine or a pharmaceutically acceptable salt or ester thereof and ergothioneine or a pharmaceutically acceptable salt or ester thereof, or subsets comprising one or more of the components of the serum or other additional components may be combined with the idebenone or a derivative thereof, N-acetyl-S-farnesyl-L-cysteine or a pharmaceutically acceptable salt or ester thereof and ergothioneine or a pharmaceutically acceptable salt or ester thereof. In some embodiments, the mixing is through mechanical means and is carried out at room temperature (approximately 20-25° C.) under a neutral pH (approximately 7.0). However, because the pH of the skin is approximately 5.5, in some embodiments the resulting formulation will have a pH value of between 4.0 and 7.0 or between 5.0 and 6.0. As persons of ordinary skill in the art are aware, pH adjusting agents exist, such as adipic acids, glycines, citric acids, calcium hydroxides, magnesium aluminometasilicates, and buffers, and these agents may be used to adjust the pH of the formulation.

Optionally, the formulation may contain one or more vitamins or one more compounds that impart a desired sun protection factor or UV filter such as p-aminobenzoic acid and its salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propylene glycol esters); cinnamic acid derivatives (menthyl and benzyl esters, α-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzylacetone and benzylacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof.

In some embodiments, the present invention also comprises formulations that in addition to comprising idebenone or a derivative thereof, N-acetyl-S-farnesyl-L-cysteine or a pharmaceutically acceptable salt or ester thereof and ergothioneine or a pharmaceutically acceptable salt or ester thereof, further comprises an additional antioxidant or salt or ester thereof, examples of which include, but are not limited to ascorbic acid (vitamin C) and its salts, ascorbyl esters of fatty acids, ascorbic acid derivatives (e.g., magnesium ascorbyl phosphate, sodium ascrobyl phosphate, and ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, lipoic acid, amines (e.g., N,N-diethylhydroxylamine and amino-guanidine), sulfhydryl compounds (e.g., glutathione), dihydroxy fumaric acid and it salts, glycine pidolate, arginine pilolate, nordihydroguaiaretic acid, bioflavinoids, curcumin, lyseine, methionine, proline, superoxide dismutase, silymarin, tea extracts, grape skin and seed extracts, melanin, and rosemary extracts.

Additionally or alternatively, the formulation may further comprise an analgesic or anti-inflammatory compound such as ibuprofen, diclofenac, capsaicin, salicylates, ketoprofen, felbinac, piroxicam, corticosteroids and NSAIDs (non-steroid anti-inflammatory drugs). Examples of corticosteroids include but are not limited to glucocorticosteroids, betamethasone, budesonide, cortisone, dexamethasone, hydrocortisone, methylprednisolone, prednisolone, predinose, and triamcinolone.

Other formulations may further comprise an anti-fungal agent. Examples of anti-fungal agents include but are not limited to amphotericin B, candicidin, dermostatin, filipin, fungichromin, hachimycin, hamycin, lucensomycin, mepartricin, natamycin, nystatin, pecilocin, perimycin, azaserine, griseofulvin, oligomycins, neomycin, pyrroinitrin, siccanin, tubercidin, viridin, butenafine, naftifine, terbinafine, bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole, sertaconazole, sulconazole, tioconazole, tolciclate, tolindate, tolnaftate, fluconawle, itraconazole, saperconazole, terconazole, acrisorcin, amorolfine, biphenamine, bromosalicylchloranilide, buclosamide, calcium propionate, chlorphenesin, ciclopirox, cloxyquin, coparaffinate, diamthazole, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Other formulations may further comprise an anti-viral agent. Examples of anti-viral agents include, but are not limited to, acyclovir, cidofovir, cytarabine, dideoxyadenosine, didanosine, edoxudine, famciclovir, floxuridine, ganciclovir, idoxuridine, inosine pranobex, lamivudine, madu, penciclovir, sorivudine, stavudine, trifluridine, valacyclovir, vidarabine, zaicitabine, zidovudine, acemannan, acetylleucine, amantadine, amidinomycin, delavirdine, foscamet, indinavir, interferon-α, interferon-β, interferon-γ, kethoxal, lysozyme, methisazone, moroxydine, nevirapine, podophyllotoxin, ribavirin, rimantadine, ritonavir2, saquinavir, stailimycin, statolon, tyromantadine, zidovudine (AZT) and xenazoic acid.

Other formulations may include antipruritc agents. Suitable antipruritic agents include, without limitation, pharmaceutically acceptable salts of methdilazine and trimeprazine.

Other formulations may include anesthetic drugs. Non-limiting examples of anesthetic drugs that are suitable for use in the context of the present invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

Other formulations may include anesthetic drugs anti-protozoal agents. Anti-protozoal agents are a group of chemical substances having the capacity to inhibit the growth of or to destroy protozoans used chiefly in the treatment of protozoal diseases. Examples of antiprotozoal agents, include but are not limited to pyrimethamine (Daraprim®), sulfadiazine, and Leucovorin.

Still further or in a further alternative, the formulation may comprise an miRNA (microRNA) or siRNA (short interfering ribonucleic acid). An siRNA is a duplex molecule that is formed from one strand that forms a hairpin or from two separate strands. Overhangs are optional, and if present, typically have six or fewer nucleotides and may be present at either end of an oligonucleotide strand. The duplex region is typically 18-30 base pairs in length and within it, there is usually perfect complementarity or complementarity except for one to four mismatches. A microRNA is typically single stranded and 17-25 nucleotides long. For example, one or more siRNAs that target IL-6 and/or TNF-α may be included.

The proceeding paragraphs describe additional active ingredients that may optionally be present in the formulations of the present invention. One or more of these agents may be included, and if included are preferably present in an effective amount.

The resulting formulation is preferably in a form that permits topical application, e.g., a cream or ointment. Use of the topical composition may comprise applying it at regular or irregular intervals to one's skin. The application may for example be applied 3-14 times per week, daily, or twice daily to one's face. In some embodiments it is applied for at least one week, at least two weeks, at least thirty days or at least six months. A person may for example, use his or her hands or an application device such as a cloth or brush to apply the formulation. As noted above the formulations of the present invention may be used to treat inflammation. The term “treat” refers to the abrogating, substantially inhibiting, slowing or reversing the progression of a condition, substantially ameliorating symptoms, protecting from harmful stimuli or generally promoting health.

Through application of the topical composition, one may provide protection against environmental stressors and may repair certain damage. Environmental stressors, the impact of which may be mitigated, may include, but are not limited to, radiation, such as UVA radiation, UVB radiation, gamma radiation, infra-red radiation, chemicals, pollution, cigarette smoke, motor vehicle emissions, certain lotions, certain cosmetics, heat, cold, wind, allergens, viruses, bacteria, fungi and other inflammation causing agents. The types of damage that may be repaired include, but are not limited to, inflammation, reddening of the skin, browning of the skin, wrinkling of the skin and DNA damage. One method by which the formulations of the present invention work is free radical scavenging. However, the present invention is not limited by the type of damage that compositions mitigate or prevent.

Any of the features of the various embodiments described herein can be used in conjunction with features described in connection with any other embodiments disclosed unless otherwise specified. Thus, features described in connection with the various or specific embodiments are not to be construed as not suitable in connection with other embodiments disclosed herein unless such exclusivity is explicitly stated or implicit from context.

TABLE 1
Serum formula

| | EXAMPLE OF | EXAMPLE OF |
|---|---|---|
| COMPOUND | AMOUNT, wt % | RANGES |
| GRANSIL SI-W 026 | 20.310 | 19.500-21.000 |
| GRANSIL SI-W 050 | 6.770 | 6.000-8.000 |
| GRANSURF 67 | 1.000 | 0.500-2.000 |
| X-25-9007A | 3.840 | 3.000-4.000 |
| SILSOFT 034 | 1.420 | 0.750-3.000 |
| VITAMIN E ACETATE | 0.200 | 0.050-0.400 |
| RETINYL LINOLEATE | 0.100 | 0.050-0.400 |
| UNISOOTH PN-47 | 2.000 | 0.500-4.000 |
| ARAZINE | 0.400 | 0.200-0.600 |
| PROTASORB L-20 | 0.200 | 0.050-0.400 |
| WATER | 14.618 | 10.000-20.000 |
| DISODIUM EDTA | 0.100 | 0.050-0.400 |
| BUTYLENE GLYCOL | 2.000 | 0.500-4.000 |
| METHYL PARABEN | 0.156 | 0.050-0.250 |
| POTASSIUM SORBATE | 0.300 | 0.100-0.500 |
| BENZOPHENONE-4 | 0.050 | 0.001-0.100 |
| UREA | 0.300 | 0.100-0.500 |
| LUMULSE PEG 3350 | 2.000 | 0.500-4.000 |
| LIPONIC EG-1 | 1.000 | 0.250-3.000 |
| MARIMOIST | 5.000 | 2.000-8.000 |
| SUBERLIFT | 3.000 | 1.000-5.000 |
| THIOTAINE | 1.000 | 0.250-3.000 |
| BONT-L PEPTIDE | 5.000 | 2.000-8.000 |
| CHRONOLINE | 2.500 | 1.000-5.000 |
| HYDROXYPROLISILANE CN | 4.000 | 2.000-6.000 |
| BETA WHITE | 5.000 | 2.000-8.000 |
| DISAPORE 20 | 0.100 | 0.025-0.300 |
| GLYCERIN | 0.500 | 0.010-0.800 |
| IDEBENONE ESTER | 1.340 | 0.500-3.000 |
| HYDROLITE 5 | 4.000 | 1.000-8.000 |
| ARISTOFLEX AVC | 0.220 | 0.050-0.500 |
| AQUAFLEX XL-30 | 3.000 | 1.000-8.000 |
| PHENONIP XB | 1.000 | 0.002-2.000 |
| FRAGRANCE UP183231/100 | 0.500 | 0.001-2.000 |
| CUPL PIC | 0.500 | 0.001-2.000 |
| SIMULGEL 600 | 2.000 | 0.001-5.000 |
| WATER | 1.000 | 0.001-4.000 |
| KTZ SUPERB SILVER | 0.500 | 0.0001-2.000 |
| KTZ FINE WHITE | 0.250 | 0.0001-2.000 |
| FD&C RED #4 IN 0.1% | 2.726 | 0.050-5.000 |
| SOLUTION IN PG/WATER | | |
| FD&C YELLOW #5, 0.1% | 0.100 | 0.0001-2.000 |
| SOLUTION IN PG/WATER | | |

### EXAMPLES

### Example 1

### Method of Making Blend and Measuring Effectiveness

Idebenone and ergothioneine were obtained from Apin Chemicals Ltd (Abingdon, Oxon, United Kingdom) and Barnet Products Corp. (Englewood Cliffs, N.J.), respectively. Arazine™ was synthesized at Signum Biosciences, Inc. (Monmouth Junction, N.J.) according to methods as described in U.S. patent publication no. 2009-0192332 A1, published Jul. 30, 2009. All chemicals were analyzed by LC/MS (Agilent 1100), ¹H and ¹³C NMR (500 MHz and 125 MHz, Bruker) for structural identity and confirmed to be >95% purity by analytical HPLC (Agilent 1200; Santa Clara, Calif.). All other agents were purchased from Sigma Chemical Co. (St. Louis, Mo.). Organic solvents were purchased from Fisher Scientific (Hampton, N.H.).

In order to evaluate the impact of UV-induced inflammation in human skin, primary cell monolayer cultures were exposed to different types of UV radiation, i.e. UVB and UVA. Primary Human Dermal Fibroblasts (HDFs) were irradiated with UVA (350 nm) and Normal Human Epidermal Keratinocytes cells (NHEKs) with UVB (305 nm). HDFs were cultured in Dulbecco's Modified Eagle Medium (DMEM) basal medium (Life Technologies, Grand Island, N.Y.), supplemented with 10% v/v fetal bovine serum (FBS), (referred to as supplemented media) at 37° C. with 5% CO₂ NHEKs were cultured in Epilife® basal medium (Life Technologies, Grand Island, N.Y.), supplemented with 60 μM calcium, 0.2% v/v bovine pituitary extract (BPE), 5 μg/ml bovine insulin, 0.18 μg/ml hydrocortisone, 5 μg/ml bovine transferrin, 5 μg/ml, 0.2 ng/ml human epidermal growth factor (referred to as supplemented media) at 37° C. with 5% CO₂.

In order to avoid possible immunomodulating effects of these agents during compound treatments, cells were kept in DMEM or Epilife® basal medium without growth supplements (referred to as depleted media). Cells were plated at a concentration of 3.8×10⁴ cells/well in supplemented media in 24-well plates. After cells were allowed to adhere (24 hours), media was changed to depleted media.

After 24 hours, depleted media was removed and fresh depleted media containing 0.3, 1.0 μM final concentrations at 0.1% v/v of idebenone, Arazine™, ergothioneine, kinetin, lipoic acid, coenzyme Q10, vitamin C, vitamin E was placed in duplicate wells. For “blend” testing, a formulation of idebenone, Arazine™ and ergothioneine (1:1:1 molar ratio) was prepared before mixing to medium. Six hours after pre-treatments, to induce a pro-inflammatory response, pre-treatment media was removed and replaced with phosphate-buffered saline (PBS). Cells were exposed to single wavelength UVA (350 nm, 12.5 J/cm²) or UVB (305 nm, 25 mJ/cm²) using a Daavlin UV Research Unit (Bryan, Ohio) in the absence of compounds without the plastic lid.

Immediately after UV irradiation, cells were placed in fresh depleted medium for 24 hours. Cell cultures were examined for viability by the reduction of 3-(4,5-dimethylthiazol-2-yl)-5-(3-carboxymethoxyphenyl)-2-(4-sulfophenyl)-2H-tetrazolium (MTS assay; Promega, Madison, Wis.) to determine the percentage of viable cells after compound and UV irradiation. Media supernatants were harvested from non-irradiated and UV irradiated cells and assayed by enzyme-linked immunosorbent assays (ELISA) for the stimulated release of pro-inflammatory mediators. NHEKs were assayed for concentrations of Interleukin-6 (IL-6) and Tumor Necrosis Factor-alpha (TNF-alpha) using appropriate protein standards (BD Biosciences; San Jose, Calif.). HDFs were assayed for concentrations of IL-6 (BD Biosciences) or pro-Matrix Metalloproteinase-1 (pro-MMP-1) using appropriate protein standards (R&D Systems; Minneapolis, Minn.).

### Example 2

### Pre-Mix v. Post-Mix Blend

NHEK cells were cultured in medium containing idebenone, N-acetyl-S-farnesyl-L-cysteine and ergothioneine at a concentration from 1 μM to 0.3 μM followed by irradiation with UVB (25 mJ/cm²) to assess whether when applied to cells, the compounds had an additive effect. Control groups contained non-irradiated cells. Twenty four hours later, cell medium were assayed for the presence of pro-inflammatory mediators by ELISA. As shown in Table 2, when the three compounds were pre-mixed and then added to the cells together, they produced a synergistic inhibition effect for the expression of IL-6 and TNFα induced by UVB. This was not observed when the three compounds were added one by one sequentially to the cells (“post-mix blend”). Surprisingly, the “post-mix blend” with all three compounds was worse than the individual compound's activity for two of the three compounds against IL-6 and for all three components against TNFα. Thus, when formulated and applied together, idebenone, N-acetyl-S-farnesyl-L-cysteine and ergothioneine demonstrate significantly greater anti-inflammatory properties, than when applied separately.

TABLE 2

| Compound | IL-6 | TNFα |
|---|---|---|
| (0.1 μM) | (% inhibited) | (% inhibited) |
| Arazine ™ | 28 | 33 |
| Idebenone | 15 | 47 |
| Ergothioneine | 12 | 35 |
| “pre-mix blend” | 58 | 69 |
| “post-mix blend” | 14 | 20 |

### Examples 3-10

In examples 3-10, the inventors investigated the anti-oxidant potential of a blend of idebenone, ergothioneine and N-acetyl-farnesyl-cysteine (the “blend”), and other commonly used anti-aging ingredients using the five Environmental Protection Factor (EPF) assays (D. H. McDaniel et al. 2005 JCD, 4:10-17). In addition, the inventors broadened the scope of the EPF to measure the inflammatory protection factor (IPF).

Cultured primary normal human keratinocytes (NHEKs) and human dermal fibroblasts (HDFs) were exposed to UVA and UVB irradiation upregulated the production of IL-6, TNFα and pro-MMP-1. Using a 180 point scoring system (20 points for each endpoint measured), results demonstrate that the blend at 165 is superior to all other ingredients tested in providing anti-oxidant protection (thereby having an anti-oxidant effect) and mitigating UV-induced pro-inflammatory cytokines and MMP production. Other ingredients tested included ferulic acid (130), vitamin E (95), lipoic acid (94), ubiquinone (87), vitamin C (73) and kinetin (72). Given the strong anti-inflammatory and anti-oxidant properties of the blend, the blend provides an optimal combination of ingredients to protect against skin aging caused by environmental damage.

### Example 3

### Radical Scavenging Capacity

This assay analyzed the overall antioxidant capacity of test ingredients for the ability to inhibit the oxidation of 2,2′-azino-bis(3-ethylbenzothiazoline-6-sulphonic acid) (ABTS). to ABTS.⁺ by metmyoglobin. The testing involved the suppression of optical density (OD) at 750 nm to a degree that is proportional to an effective oxidation inhibition. The EPF scoring system was based on the lowest effective concentration as previously described by MacDaniel et al., 2005 and scores were assigned as follows: 10⁻³ M=5; 10⁻⁴ M=15; 10⁻⁵ M=20. The results are provided in Table 3 below:

TABLE 3

| | Effective and | |
|---|---|---|
| | oxidant conc | |
| Ingredient(s) | (mM) | EPF Score |
| Blend | 0.10 | 15 |
| Ferulic acid | 0.03 | 20 |
| Kinetin | >3 | 5 |
| Lipoic acid | >3 | 5 |
| Ubiquinone | >3 | 5 |
| Vitamin C | 0.03 | 20 |
| Vitamin E | 0.06 | 20 |

### Example 4

### LDL Pro-Oxidative System Measuring Primary Oxidation by-Products

Human LDL was oxidized with or without 0.1 mM materials under a CuSO₄ oxidative system in Ham's F-10 medium. Lipid peroxidation was quantified after 24 hours incubation with test ingredients measuring chloroform-extracted hydroperoxides directly utilizing a redox reaction with the production of ferric ions from ferrous ions, detected using thiocyanate ion as a chromogen and expressed as the change in optical density (OD) at 500 nm to a degree that is proportional to an effective peroxidation inhibition based on a standard hydroperoxide solution. The results are provided in Table 4 below:

TABLE 4

| | Lipid hydrogen- | |
|---|---|---|
| Ingredient(s) | peroxide % | |
| (100 μM) | protection | EPF Score |
| Blend | 44 | 10 |
| Ferulic acid | 94 | 20 |
| Kinetin | 1 | 1 |
| Lipoic acid | 5 | 2 |
| Ubiquinone | 14 | 5 |
| Vitamin C | 0 | 1 |
| Vitamin E | 56 | 12 |

### Example 5

### Microsome Pro-Oxidative System Measuring Secondary Oxidation by-Products

Rat liver microsomes were oxidized with or without test ingredients at 0.1 mM using a NADPH/ADP/Fe³⁺ oxidative system. Secondary oxidation products were quantified after 24 hours incubation by measuring MDA equivalents directly utilizing the production of Thiobarbituric acid-reactive substances (TBARS). Results are expressed as the change in optical density (OD) at 532 nm to a degree that is proportional to an effective oxidation inhibition based on 1,1,3,3-tetraethoxypropane as MDA-TBA production standard. The results are provided in Table 5 below:

TABLE 5

| Ingredient(s) | Lipid MDA % | |
|---|---|---|
| (100 μM) | protection | EPF Score |
| Blend | 87 | 20 |
| Ferulic acid | 63 | 17 |
| Kinetin | 0 | 0 |
| Lipoic acid | 25 | 7 |
| Ubiquinone | 62 | 17 |
| Vitamin C | 7 | 2 |
| Vitamin E | 45 | 12 |

### Example 6

### UVB Induced DNA Damage (Thymine Dimer Formation)

Primary Human Epidermal Keratinocytes (NHEKs) were cultured in the presence of each test ingredient (1 μM) for 6 hours and later removed prior to UVB irradiation (200 mJ/cm²). Cells were fixed after 1 hour and stained with anti-thymine dimer antibody. Positive rounded stained cells were counted using a fluorescence microscope. No cell cytotoxicity of NHEKs was observed. The results are provided in Table 6 below:

TABLE 6

| Ingredient(s) | DNA damage % | |
|---|---|---|
| (1 μM) | protection | EPF Score |
| Blend | 56 | 20 |
| Ferulic acid | 14 | 5 |
| Kinetin | 14 | 5 |
| Lipoic acid | 13 | 5 |
| Ubiquinone | 9 | 3 |
| Vitamin C | 16 | 6 |
| Vitamin E | 9 | 3 |

### Example 7

### UVB Induced Sun Burn Cell (SBC) Formation Using EpiDerm™

EpiDerm™ inserts (MatTek®) were topically treated in the presence of each test ingredient for 6 hours, after which they were removed, inserts were irradiated with 200 mJ/cm² UVB, and they fixed for H&E staining after 24 hours. The number of sun burn cells (SBCs) (white arrows) was counted. All compounds were tested at 1 mM. No cytotoxicity of 3D skin was observed at concentrations shown. The results are provided in Table 7 below:

TABLE 7

| Ingredient(s) | | |
|---|---|---|
| (1 mM) | SBC % inhibition | EPF Score |
| Blend | 72 | 20 |
| Ferulic acid | 64 | 18 |
| Kinetin | 62 | 18 |
| Lipoic acid | 27 | 8 |
| Ubiquinone | 46 | 13 |
| Vitamin C | 31 | 9 |
| Vitamin E | 21 | 6 |

### Example 8

### UVB Induced Pro-Inflammatory Cytokine Release (IL-6 and TNFα)

To test these anti-oxidants for anti-inflammatory properties, primary Human Epidermal Keratinocytes (NHEKs) were cultured in the presence of each ingredient for 6 hours. Ingredients were then removed and cells were irradiated with 25 mJ/cm2 UVB. Media supernatants were collected after 24 hours and analyzed by ELISA for IL-6 and TNF-α. All ingredients were tested at 1 μM. No cell cytotoxicity of NHEKs was observed at concentrations shown. The results are provided in Table 8 below:

TABLE 8

| Ingredient(s) | IL-6 (% | | TNFα (% | |
|---|---|---|---|---|
| (1 μM) | inh) | EPF Score | inh) | EPF Score |
| Blend | 66 | 20 | 77 | 20 |
| Ferulic acid | 54 | 16 | 55 | 14 |
| Kinetin | 53 | 16 | 39 | 10 |
| Lipoic acid | 46 | 14 | 52 | 14 |
| Ubiquinone | 34 | 10 | 48 | 12 |
| Vitamin C | 13 | 4 | 36 | 9 |
| Vitamin E | 12 | 4 | 16 | 4 |

### Example 9

### UVA Induced IL-6 and MPP-1 Production

To test these anti-oxidants for anti-inflammatory and anti-aging properties, primary Human Dermal Fibroblasts (HDFs) were cultured in the presence of each ingredient for 6 hours. Later, ingredients were removed and cells were irradiated with 12.5 J/cm² UVA. Media supernatants were collected after 24 hours and analyzed by ELISA for IL-6 and pro-MMP-1. All ingredients were tested at 1 μM. No cell cytotoxicity of HDFs was observed at concentrations shown. The results are provided in Table 9 below:

TABLE 9

| Ingredient(s) | IL-6 (% | | MMP-1 (% | |
|---|---|---|---|---|
| (1 μM) | inh) | EPF Score | inh) | EPF Score |
| Blend | 90 | 20 | 99 | 20 |
| Ferulic acid | 62 | 13 | 37 | 7 |
| Kinetin | 68 | 15 | 11 | 2 |
| Lipoic acid | 62 | 13 | 80 | 16 |
| Ubiquinone | 79 | 17 | 26 | 5 |
| Vitamin C | 81 | 17 | 13 | 3 |
| Vitamin E | 82 | 18 | 80 | 16 |

### Example 10

### Summary of Total EPF Scores

Table 10 below provides a summary of the EPF scores. (The numbers in the header refer to the examples from which the data were obtained.) As the table illustrates: (1) the blend provides the best protection amongst tested ingredients against skin aging caused by environmental damage; (2) the blend possesses the strongest tested anti-inflammatory activity protecting against UVA and UVB-induced damage to skin cells; and (3) the blend yields the highest Environmental Protection Factor (EPF), thus representing a promising new product for skin care protection.

TABLE 10

| Assay | 3 | 4 | 5 | 6 | 7 | 8A | 8B | 9A | 9B | TTL |
|---|---|---|---|---|---|---|---|---|---|---|
| Blend | 15 | 10 | 20 | 20 | 20 | 20 | 20 | 20 | 20 | 165 |
| Ferulic acid | 20 | 20 | 17 | 5 | 18 | 16 | 14 | 13 | 7 | 130 |
| Kinetin | 5 | 1 | 0 | 5 | 18 | 16 | 10 | 15 | 2 | 72 |
| Lipoic acid | 5 | 2 | 7 | 15 | 8 | 14 | 14 | 13 | 16 | 94 |
| Ubiquinone | 5 | 5 | 17 | 3 | 13 | 10 | 12 | 17 | 5 | 87 |
| Vitamin C | 20 | 1 | 2 | 6 | 9 | 4 | 9 | 17 | 3 | 73 |
| Vitamin E | 20 | 12 | 12 | 3 | 6 | 4 | 4 | 18 | 16 | 95 |

We claim:

1. A formulation comprising: idebenone or a derivative thereof, N-acetyl-S-farnesyl-L-cysteine or a pharmaceutically acceptable salt or ester thereof and ergothioneine or a pharmaceutically acceptable salt or ester thereof.

2. The formulation of claim 1, wherein
(a) the idebenone or the derivative thereof is present in an amount of 0.0001-5.0 wt. % based on the total weight of the formulation;
(b) the N-acetyl-S-farnesyl-L-cysteine or the pharmaceutically acceptable salt or ester thereof is present in an amount of 0.0001-10.0 wt. % based on the total weight of the formulation; and
(c) the ergothioneine or the pharmaceutically acceptable salt or ester thereof is present in an amount of 0.0001-10.0 wt. % based on the total weight of the formulation.

3. The formulation of claim 2, wherein the idebenone or the derivative thereof is:

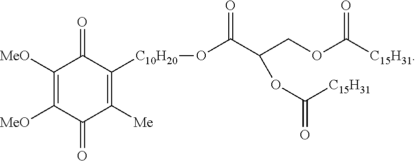

4. A method for preventing a skin change, said method comprising applying a topical preparation to the skin, the preparation comprising an effective amount of idebenone or a derivative thereof, N-acetyl-S-farnesyl-L-cysteine or a pharmaceutically acceptable salt or ester thereof and ergothioneine or a pharmaceutically acceptable salt or ester thereof.

5. A method of inhibiting UV induction of pro-inflammatory cytokines, said method comprising administering a formulation of claim 1 to an organism, wherein said organism has been exposed to UV radiation.

6. The method of claim 5, wherein said administering is by topically applying the formulation to the organism.

7. The method of claim 6, wherein the formulation is applied to a region of skin of the organism.

8. The method of claim 5, wherein the cytokines comprise IL-6 or TNF-α or a combination thereof.

9. A method for reducing sun burn cell formation, said method comprising administering an effective amount of a formulation of claim 1 to an organism, wherein said organism has been exposed to UV radiation.

10. A method for treating inflammation comprising administering an effective amount of a formulation according to claim 1 to a person in need thereof.

11. A method for repairing DNA damage comprising administering an effective amount of a formulation according to claim 1 to a person in need thereof.

12. A method for treating human skin comprising topically applying to the human skin an effective amount of a composition comprising the formulation of claim 1, wherein said treating comprises scavenging for free radicals.

13. A method for treating human skin comprising topically applying to the human skin an effective amount of a composition comprising the formulation of claim 1, wherein said treating comprises an anti-oxidant effect.

14. The method according to claim 13, wherein said anti-oxidant effect protects against skin aging.

15. A method for treating human skin comprising topically applying to the human skin an effective amount of a composition comprising the formulation of claim 1, wherein said treating mitigates against at least one of UV-induced pro-inflammatory cytokines and MMP production.

16. The method according to claim 15, wherein said treating mitigates against both of UV-induced pro-inflammatory cytokines and MMP production.
